(19) United States
(12) Patent Application Publication
(10) Pub. No.: US 2020/0283832 A1
(43) Pub. Date: Sep. 10, 2020

(54) SINGLE NUCLEOTIDE ANALYTICAL METHOD AND ASSOCIATED PROBES

(71) Applicant: BASE4 INNOVATION LIMITED, Cambridge, Cambridgeshire (GB)

(72) Inventors: Barnaby BALMFORTH, Cambridge, Cambridgeshire (GB); Cameron Alexander FRAYLING, Cambridge, Cambridgeshire (GB)

(73) Assignee: BASE4 INNOVATION LTD, Cambridge, Cambridgeshire (GB)

(21) Appl. No.: 16/758,217

(22) PCT Filed: Oct. 23, 2018
(86) PCT No.: PCT/EP2018/078991

§ 371 Date: Apr. 22, 2020

(30) Foreign Application Priority Data
Oct. 23, 2017 (EP) 17197878.6

(51) Int. Cl. C12Q 1/6823; C12Q 1/48

(52) U.S. Cl. CPC C12Q 1/6823; C12Q 1/48

(57) ABSTRACT

A method of detecting whether the nucleobase of a given nucleoside triphosphate molecule in an analyte includes a given chemical or structural modification comprising (1) reacting the analyte in the presence of a polymerase with a biological probe comprised of (a) a pair of single-stranded first oligonucleotides each comprising an exonuclease blocking-site; a restriction endonuclease recognition-site; a single nucleotide capture-site and respectively first and second fluorophore(s) in a substantially undetectable state, and (b) at least one second single-stranded oligonucleotide to create a used-probe duplex; (2) reacting the duplex with a restriction endonuclease system comprised of at least one restriction endonuclease; (3) creating another used-probe duplex; (4) digesting the first oligonucleotide elements with an enzyme having 5′-3′ exonucleolytic activity to produce fluorophore(s) in a detectable state; and (5) detecting the fluorophore(s) released.

This invention relates to a method and associated biological probes for detecting modifications in nucleobases of inter alia naturally-occurring DNA and RNA.

In our previous patent application WO2015121675 we have described the use of a three-component biological probe comprising a first component having single- and double-stranded oligonucleotide regions and a pair of second components including different fluorophores in a quenched state to identify the methylation status of a given single nucleotide in a polynucleotide analyte. In this method, the used-probe is cleaved by a methylation-dependent or a methylation-sensitive restriction endonuclease prior to the release of the fluorophores in an unquenched state by exonucleolysis.

We have now developed another version of this method which is not only more widely applicable to a range of nucleobase modifications and the restriction endonucleases currently available but is much simpler, more sensitive and generates both a stronger signal and higher signal to noise ratio. Thus, according to a first aspect of the invention there is provided a method of detecting whether the nucleobase of a given nucleoside triphosphate molecule in an analyte does or does not include a given chemical or structural modification characterised by the steps of (1) reacting the analyte in the presence of a polymerase with a biological probe comprised of (a) a pair of single-stranded first oligonucleotides each comprising an exonuclease blocking-site; at least one restriction endonuclease recognition-site located on the 5′ side of the blocking-site; a single nucleotide capture-site located within the recognition-site and respectively first and second fluorophore(s) located on the 5′ side of the recognition-site arranged so as to be substantially undetectable and (b) at least one second and optionally at least one third single-stranded oligonucleotide each separate from the first oligonucleotide and capable of hybridising to complementary flanking regions on the 3′ and 5′ sides of the capture-site of one, other or both of the first oligonucleotides in the pair to create a used-probe duplex consisting of (b1) one or other of the first oligonucleotides in the pair and (b2) a component comprised of the second oligonucleotide, one or other of a modified or unmodified nucleotide derived from the nucleoside triphosphate molecule and optionally the third oligonucleotide wherein the duplex is comprised of one of the four possible (b1)/(b2) duplex permutations; (2) reacting the duplex produced in step (1) with a restriction endonuclease system comprised of at least one restriction endonuclease adapted to selectively cleave the (b1) strand at the recognition-site to create depending on the presence or absence of the modification in the original nucleoside triphosphate molecule (i) only exonucleolytically-digestible first oligonucleotide elements bearing first fluorophore(s) or (ii) only exonucleolytically-digestible first oligonucleotide elements bearing second fluorophore(s) or (iii) a mixture of exonucleolytically-digestible first oligonucleotide elements respectively bearing first and second fluorophore(s); (3) creating another used-probe duplex from the (b2) component and another first oligonucleotide in the pair and thereafter iterating steps (2) and (3); (4) digesting the first oligonucleotide elements with an enzyme having 3′-5′ exonucleolytic activity to produce fluorophore(s) in a detectable state after either or both of steps (2) or (3) and (5) detecting the fluorophore(s) released in step (4) and inferring from the nature of the fluorescence signal observed whether the original single nucleoside triphosphate molecule was modified or unmodified.

In a second, related aspect of the invention there is provided a method of detecting whether the nucleobase of a given nucleoside triphosphate molecule in an analyte does or does not include a given chemical or structural modification characterised by the steps of (1) reacting the analyte in the presence of a polymerase with a biological probe comprised of (a) a pair of single-stranded first oligonucleotides each comprising an exonuclease blocking-site; at least one restriction endonuclease recognition-site located on the 3′ side of the blocking-site; a single nucleotide capture-site located within the recognition-site and respectively first and second fluorophore(s) located on the 3′ side of the recognition-site arranged so as to be substantially undetectable and (b) at least one second and optionally at least one third single-stranded oligonucleotide each separate from the first oligonucleotide and capable of hybridising to complementary flanking regions on the 3′ and 5′ sides of the capture-site of one, other or both of the first oligonucleotides in the pair to create a used-probe duplex consisting of (b1) one or other of the first oligonucleotides in the pair and (b2) a component comprised of the second oligonucleotide, one or other of a modified or unmodified nucleotide derived from the nucleoside triphosphate molecule and optionally the third oligonucleotide wherein each duplex is comprised of one of the four possible (b1)/(b2) duplex permutations; (2) reacting the duplex produced in step (1) with a restriction endonuclease system comprised of at least one restriction endonuclease adapted to selectively cleave the (b1) strand at the recognition-site to create depending on the presence or absence of the modification in the original nucleoside triphosphate molecule (i) only exonucleolytically-digestible first oligonucleotide elements bearing first fluorophore(s) or (ii) only exonucleolytically-digestible first oligonucleotide elements bearing second fluorophore(s) or (iii) a mixture of exonucleolytically-digestible first oligonucleotide elements respectively bearing first and second fluorophore(s); (3) creating another used-probe duplex from the (b2) component and another first oligonucleotide in the pair and thereafter iterating steps (2) and (3); (4) digesting the first oligonucleotide elements with an enzyme having 5′-3′ exonucleolytic activity to produce fluorophore(s) in a detectable state after either or both of steps (2) and (3); and (5) detecting the fluorophore(s) released in step (4) and inferring from the nature of the fluorescence signal observed whether the original single nucleoside triphosphate molecule was modified or unmodified.

The analyte to which this method is applicable can be conveniently prepared from a nucleic acid precursor by progressive enzymatic digestion. In one embodiment, this can be achieved by progressive exonucleolysis of the precursor followed by the action of a kinase on the single nucleoside monophosphates obtained (see for example Biotechnology and Bioengineering by Bao and Ryu (DOI 10.1002/bit.21498). Preferably, however, the nucleoside triphosphate molecules in the analyte are produced directly from the precursor by progressive pyrophosphorolysis. The nucleic acid precursor employed in this step is suitably a single- or double-stranded polynucleotide the length of which can in principle be unlimited; for example including up to the many millions of nucleotides found in a human gene or chromosome fragment. Typically, however, the polynucleotide will be at least 50, preferably at least 150 nucleotides long; suitably it will be greater than 500, greater than 1000 and in many cases thousands of nucleotides long. The nucleic acid precursor is preferably RNA or DNA of natural origin (e.g. derived from a plant, animal, bacterium or a virus) although the method can also be used to analyse completely synthetic or synthetically-produced. Included are RNA, DNA or other nucleic acids made up wholly or in part of nucleotides whose associated nucleobases are not commonly encountered in nature; i.e. nucleobases other than adenine (A), guanine (G), cytosine (C), thymine (T) and uracil (U). Examples of such nucleobases include 4-acetylcytidine, 5-(carboxyhydroxylmethyl)uridine, 2-O-methylcytidine, 5-carboxymethylaminomethyl-2-thiouridine, 5-carboxymethylamino-methyluridine, dihydrouridine, 2-O-methylpseudouridine, 2-O-methylguanosine, inosine, N6-isopentyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, 5-methoxyuridine, 5-methoxycarbonylmethyl-2-thiouridine, 5-methoxycarbonylmethyluridine, 2-methylthio-N6-isopentenyladenosine, uridine-5-oxyacetic acid-methylester, uridine-5-oxyacetic acid, wybutoxosine, wybutosine, pseudouridine, queuosine, 2-thiocytidine, 5-methyl-2-thiouridine, 2-thiouridine, 4-thiouridine, 5-methyluridine, 2-O-methyl-5-methyluridine and 2-O-methyluridine. In the case of DNA, the single nucleoside triphosphates generated are deoxyribonucleoside triphosphates whilst in the case of RNA they are ribonucleoside triphosphates.

In one embodiment of the method, generation of the analyte further comprises a first sub-step of attaching the nucleic acid precursor to a substrate. Typically, this substrate comprises a microfluidic surface, a micro-bead or a permeable membrane; for example one made of glass or a non-degradable polymer. Preferably, the substrate further comprises a surface specifically adapted to receive the nucleic acid precursor. There are many ways in which the nucleic acid precursor can be attached to such surfaces any of which can in principle be used in this sub-step. For example, one method involves priming a glass surface with a functionalised silane such as an epoxysilane, an aminohydrocarbylsilane or a mercaptosilane. The reactive sites so generated can then be treated with a derivative of the nucleic acid precursor which has been modified to include a reactive terminal amine, succinyl or thiol group.

In another embodiment, the nucleic acid precursor is pyrophosphorolysed to generate an analyte comprising a stream of single nucleoside triphosphate molecules the order of which corresponds to that of the sequence of the analyte. Such pyrophosphorolysis may be carried out at a temperature in the range 20 to 90° C.; for example in the presence of a reaction medium comprising a suitable polymerase showing such enzymatic behaviour. Preferably, it is carried out under conditions of continuous flow so that the single nucleoside triphosphate molecules are continually removed from the reaction zone as they are liberated. Most preferably, the pyrophosphorolysis is carried out by causing an aqueous buffered medium containing the enzyme and the other typical additives to flow over the surface to which the nucleic acid analyte is bound.

In yet another embodiment, the enzyme used is one which can cause progressive 3′-5′ pyrophosphorolytic digestion of the nucleic acid precursor to yield a stream of nucleoside triphosphate molecules with high fidelity and at a reasonable reaction rate. Preferably, this digestion rate is as fast as possible and in one embodiment is in the range 1 to 50 nucleoside triphosphate molecules per second. Further information about the pyrophosphorolysis reaction as applied to the digestion of polynucleotides can be found for example in J. Biol. Chem. 244 (1969) pp. 3019-3028 to which the reader is directed. Suitably, the pyrophosphorolytic digestion is carried out in the presence of a medium which further comprises for example pyrophosphate anion and magnesium cations; preferably in millimolar concentrations. After this digestion, the solution containing the released nucleoside triphosphates is suitably treated with a pyrophosphatase, to hydrolyse any residual pyrophosphate to phosphate anion.

In one embodiment, the nucleic acid precursor is obtained from cellular material present in conventional biological or medical samples using known extraction techniques including cell lysing, extraction and chromatographic separation.

In step (1), the analyte containing the nucleoside triphosphate molecule(s), is reacted in the presence of a polymerase and optionally a ligase to generate a used-probe duplex. In one embodiment where a ligase is additionally employed this duplex will comprise a discrete substantially double-stranded fourth oligonucleotide (see below) although formation of such a fourth oligonucleotide is not critical to the efficacy of the method. The nucleic acid analyte employed however is one which is expected to comprise either or both of the nucleoside triphosphate molecule(s) whose associated nucleobases are expected to be modified or unmodified. By the term ‘modified’, is meant any modification be it chemical or structural which differentiates the modified nucleobase from its unmodified pair. Such modifications may include chemical modifications such as alkylation, hydroxyalkylation, alkoxylation, amination, halination, acetylation and glucosylation although it will be appreciated that the method will be generally applicable to any modified/unmodified nucleobase pair provided that the modification does not interfere with the capture performed in this step. In one embodiment, the modification is selected from methylation, hydroxymethylation and glucosylated hydroxymethylation. In another, the unmodified nucleoside triphosphate molecule is selected from the groups consisting of those unmodified nucleoside triphosphate molecules which are the constituents of naturally-occurring DNA or RNA. In yet another the associated modification is methylation of either or both of the nucleobases cytosine and adenine.

The polymerase used in this step is suitably selected from the group consisting of those which show essentially neither exo-nor endonuclease activity under the reaction conditions. Examples of polymerases which can be advantageously used include, but are not limited to, the prokaryotic pol 1 enzymes or enzyme derivatives obtained from bacteria such as Escherichia coli (e.g. Klenow fragment polymerase), Thermus aquaticus (e.g. Taq Pol), Bacillus stearothermophilus, Bacillus caldovelox and Bacillus caldotenax. Any suitable ligase can in principle be used in this step.

The biological probe employed in step (1) is comprised of two or optionally three components; (a) a pair of single-stranded first oligonucleotides labelled with differing first and second characteristic fluorophores in an undetectable state and (b) second and optionally third unlabelled single-stranded oligonucleotides capable of hybridising to complementary flanking regions on the first oligonucleotides. In one three-component embodiment, the same second and third oligonucleotides may be capable of hybridising to both first oligonucleotides in the pair. In another embodiment, where a ligase is not employed, only the second oligonucleotide is common and a corresponding pair of third oligonucleotides are employed. In yet another embodiment, the second and third oligonucleotides are discrete entities whilst in yet another they are oligonucleotide regions linked by means of a linker-region. In this latter case, in one embodiment the linker-region links the ends of the second and third oligonucleotide regions. The linker-region can in principle be any divalent group but is conveniently itself another oligonucleotide region. In one embodiment this oligonucleotide linker-region is unable to hybridise substantially to either of the first oligonucleotides in the pair.

The separate first, second and third oligonucleotides are chosen so that in step (1) the second and optionally the third oligonucleotide(s) can hybridise respectively to 3′ side and 5′ side flanking regions on the relevant first oligonucleotide which regions themselves are juxtaposed either side of a capture-site comprising the single nucleotide whose nucleobase is complementary to that borne by the nucleoside triphosphate molecule to be detected by the probe. The capture-site is also part of a restriction endonuclease recognition-site. This makes the probe highly selective for the particular nucleoside triphosphate being investigated.

Typically, each of the first oligonucleotides in the pair is up to 150 nucleotides long, preferably between 10 and 100 nucleotides. In one embodiment, the second oligonucleotide is shorter than the complementary 3′ side flanking region of the first oligonucleotide by at least one nucleotide. In another, there is at least a single nucleotide mismatch between the 3′ end of the first oligonucleotide and the nucleotide opposite it on the second oligonucleotide to prevent the nucleoside triphosphate being captured by the polymerase at this point. Similarly, in one embodiment the third oligonucleotide is longer than the complementary 5′ side flanking region of the first oligonucleotide by at least one nucleotide, while in another there is at least a single nucleotide mismatch between the 3′ end of the third oligonucleotide and the nucleotide opposite it in the first oligonucleotide to prevent the nucleoside triphosphate being captured by the polymerase at this point.

It is a common feature of each of the first oligonucleotides in the pair that it is labelled with one or more fluorophores which are unique and characteristic of it. In both first oligonucleotides, these fluorophore(s) are arranged so as to be substantially undetectable when the probe is in an unused state. Preferably, they are arranged to be essentially non-fluorescing at those wavelengths where the fluorophores are designed to be detected. Thus, although a fluorophore may exhibit general, low-level background fluorescence across a wide part of the electromagnetic spectrum, there will typically be one or a small number of specific wavelengths or wavelength envelopes where the intensity of the fluorescence is at a maximum. It is at one or more of these maxima, where the fluorophore is characteristically detected, that essentially no fluorescence should occur. In the context of this patent, by the term ‘essentially non-fluorescing’ or equivalent wording is meant that the intensity of fluorescence of the total number of fluorophores attached to the relevant first oligonucleotide at the relevant characteristic wavelength or wavelength envelope is less than 25%; preferably less than 10%; more preferably less than 1% and most preferably less than 0.1% of the corresponding intensity of fluorescence of an equivalent number of free fluorophores.

In principle, any method can be used to ensure that in the first oligonucleotide's unused state the fluorophores are essentially non-fluorescing. In one embodiment this is achieved by arranging the fluorophores in close proximity to each other so that they quench one another (self-quenching arrangement). In another embodiment, the region containing the fluorophore(s) further includes separate quencher(s) in close proximity thereto by means of which the same outcome can be achieved. In the context of this patent, what constitutes ‘close proximity’ between fluorophores or between fluorophores and quenchers will depend on the particular fluorophores and possibly the structural characteristics of the first oligonucleotide. Consequently, it is intended that this term should be construed with reference to the required outcome rather than any particular structural arrangement of these items. However, and for the purposes of providing exemplification only, it is pointed out that when adjacent fluorophores are separated by a distance corresponding to the characteristic Forster distance (typically less than 5 nm) sufficient quenching will generally be achieved.

As regards the fluorophores themselves, they can in principle be chosen from any of those conventionally used in the art including but not limited to xanthene moieties e.g. fluorescein, rhodamine and their derivatives such as fluorescein isothiocyanate, rhodamine B and the like; coumarin moieties (e.g. hydroxy-, methyl- and aminocoumarin) and cyanine moieties such as Cy2, Cy3, Cy5 and Cy7. Specific examples include fluorophores derived from the following commonly used dyes: Alexa dyes, cyanine dyes, Atto Tec dyes, and rhodamine dyes. Examples also include: Atto 633 (ATTO-TEC GmbH), Texas Red™, Atto 740 (ATTO-TEC GmbH), Rose Bengal, Alexa Fluor™ 750 C₅-maleimide (Invitrogen), Alexa Fluor™ 532 C₂-maleimide (Invitrogen) and Rhodamine Red C₂-maleimide and Rhodamine Green as well as phosphoramadite dyes such as Quasar 570. Alternatively, a quantum dot or a near infra-red dye such as those supplied by LI-COR Biosciences can be employed and should be considered to be ‘fluorophores’ for the purposes of interpreting this patent. The fluorophore(s) are typically attached to the first oligonucleotide via a nucleobase using chemical methods known in the art.

Suitable quenchers include those which work by a Forster resonance energy transfer (FRET) mechanism. Examples of commercially available quenchers which can be used in association with the above mentioned-fluorophores include but are not limited to DDQ-1, Dabcyl, Eclipse, Iowa Black FQ and RQ, IR Dye-QC1, BHQ-0, BHQ-1, -2 and -3 and QSY-7 and -21.

It is another common feature of the first oligonucleotides that they include at least one exonuclease blocking-site. This will be located on either the 3′ or 5′ side of the recognition-site depending on which of the two methods described above is being employed. In one embodiment, the first oligonucleotide may include such blocking-sites on both sides or adjacent the 3′ or 5′ end as the case may be. In principle the blocking-site can be any region which, by virtue of its chemical constitution, renders the first oligonucleotide resistant to exonucleolysis at that point. Such regions may for example include phosphorothioate linkers, oligonucleotide spacers (e.g. Spacer3, Spacer 9, Spacer 18, dSpacer and the like), 2′-O-methyl RNA bases, inverted bases, desthiobiotin-TEG, dithiol, hexanediol, and quenchers (e.g. BHQ).

In one embodiment the exonuclease blocking-site can be achieved by rendering the first oligonucleotides circular; i.e. a closed-loop. In another, where a single-strand-specific exonuclease is employed in step (4) below, this site may comprise a double-stranded region on the first oligonucleotide(s). In yet another embodiment, the second and/or third oligonucleotides are also provided with similar exonuclease blocking-sites.

It is yet another common feature of the first oligonucleotides that they include a restriction endonuclease recognition-site which further includes the capture-site referred to above. This recognition-site is arranged on either (1) the 5′ side of the exonuclease blocking-site and the 3′ side of the fluorophore(s) or (2) the 3′ side of the exonuclease blocking-site and the 5′ side of the fluorophore(s) also depending on which of the two methods described above is employed.

In one embodiment of step (1), a set of multiple biological probes may be employed in the same method with the first oligonucleotides in each pair comprising a different nucleotide capture-site and different first and second fluorophores. In another embodiment, this set may further include other biological probes of the type taught in our European patent application 17171168.2 to which the reader is directed. For example, and by way of illustration, if the methylation state of a DNA precursor is being investigated one pair might be able to capture methylated or unmethylated deoxyadenosine triphosphate if the nucleobase associated with nucleotide capture-site is thymine and another pair methylated or unmethylated deoxycytidine triphosphate where the capture-site includes a guanine nucleobase. In embodiments such as these, where more than one first oligonucleotide pair is employed, it is preferred that each differently labelled first oligonucleotide nevertheless comprises the same recognition-site so that the minimum number of restriction endonuclease need be employed. In one embodiment, it is therefore preferred that the recognition-site will be comprised of a sequence containing at least one of each of the typical nucleotides of RNA or DNA (as the case may be) with one or other of the recognition-sites in the pair including a modified nucleobase.

Step (1) is suitably carried out by contacting each single nucleoside triphosphate in the analyte with the enzymes and one or more probes as described above at a temperature in the range 20 to 80° C.

The product of step (1) is comprised of one or more duplexes whose constituent parts are respectively (i) one of the first oligonucleotides and (ii) a component comprised of a second oligonucleotide, one or other of the nucleotide molecules (modified or unmodified) derived from the nucleoside triphosphate molecule and optionally a third oligonucleotide. As explained above, where step (1) is carried out in the presence of a ligase, the various components of (ii) will together comprise a discrete fourth oligonucleotide and the used probe will be double-stranded at least in the vicinity of the recognition-site. If the second and third oligonucleotides have previously been joined together by a linker-region then it will be readily apparent that this will lead to a fourth oligonucleotide which is a closed-loop and highly resistant to exonucleolysis.

It will likewise be apparent that in the binary case involving a nucleic acid analyte which may contain either or both modified or unmodified nucleoside triphosphate molecules and a pair of first oligonucleotides respectively bearing first and second fluorophore(s) four possible outcomes are possible. The permutation of these outcomes will comprise the first oligonucleotide bearing the first fluorophore(s) and a component comprised of the second oligonucleotide, the modified nucleoside triphosphate molecule derived from the analyte and optionally the third oligonucleotide (‘first-modified’); the first oligonucleotide bearing the first fluorophore(s) and the component comprised of the second oligonucleotide, the unmodified nucleoside triphosphate molecule derived from the analyte and optionally the third oligonucleotide (‘first-unmodified’); the first oligonucleotide bearing the second fluorophore(s) and the component comprised of the second oligonucleotide, the modified nucleoside triphosphate molecule derived from the analyte and optionally the third oligonucleotide (‘second-modified’) and the first oligonucleotide bearing the second fluorophore(s) and the component comprised of the second oligonucleotide, the unmodified nucleoside triphosphate molecule derived from the analyte and optionally the third oligonucleotide (‘second-unmodified’). Thus, in various embodiments of the methods described above, one, two, three or all four of these duplexes may be present in the product of step (1). One of ordinary skill will also understand that for more complicated analyses where multiple probes and multiple nucleoside triphosphate molecule types are present the number of permutations will correspondingly increase although the consequential set of permutations can easily be determined.

In step (2), the duplexes created in step (1) are reacted at a temperature in the range 20 to 100° C. with a restriction endonuclease system capable of differentiating between some or all of the permutations mentioned above. This differentiating, as will be explained below, is effected by the correct choice of the components of the restriction endonuclease system and the characteristics of the corresponding restriction endonuclease recognition site(s). In one embodiment, this restriction endonuclease system includes at least one nicking restriction endonuclease designed to cleave only the component derived from the first oligonucleotide at its recognition-site. In another embodiment, the restriction endonuclease may be one able to cut both strands and the second and/or third oligonucleotide may be rendered resistant to cleavage for example by inclusion of endonucleolytic blocking-groups in either or both of the second and third oligonucleotides. In one embodiment, these blocking-groups may be selected from phosphorothioate linkages and other backbone modifications commonly used in the art, oligonucleotide spacers, phosphate groups, or the like. In a third embodiment, where no ligase is employed, the restriction endonuclease may be one able to cut both components at a cutting site remaining between the second and third oligonucleotides after the capture of the nucleoside triphosphate molecule.

Details of suitable restriction endonucleases, including nicking endonucleases which can be used with the method, probes and probe systems of the present invention can be found at http://rebase.neb.com in the database associated therewith.

In one embodiment, the restriction endonuclease system comprises a first restriction endonuclease able only to cleave ‘first-modified’ duplexes and a second restriction endonuclease able only to cleave ‘second-unmodified’ duplexes. In this case, only first fluorophore(s) will be released if the nucleoside triphosphate molecule is modified and only second fluorophore(s) if it is unmodified. In this embodiment, the pair of restriction endonucleases which may be employed includes DpnI and Hpy188l if the modification under investigation is adenine methylation.

In another embodiment, the restriction endonuclease system comprises a restriction endonuclease unable to cleave ‘first-modified’ duplexes. In this case only second fluorophore(s) will be released if the nucleoside triphosphate molecule is modified and both first and second fluorophore(s) if it is not. In this example, the restriction endonuclease may be for example Clal or Alwl if the modification is adenine methylation.

In another embodiment, the restriction endonuclease system comprises a first restriction endonuclease capable of cleaving all of the duplexes and a second restriction endonuclease able to cleave only ‘second-unmodified’ duplexes. In this case, the recognition-site of the second oligonucleotide further includes a restriction endonuclease blocking-site at the site of cleavage by the first restriction endonuclease, which is not in the same position as the site of cleavage by the second restriction endonuclease. Here, only first fluorophore(s) will be released if the nucleoside triphosphate is modified and both first and second fluorophore(s) if it is not. One example of this approach as applied to adenine methylation is the use of the restriction endonucleases BfuCl and BspEl in combination with a second oligonucleotide including a phosphorothioate blocking linkage at the cleavage site of BfuCl.

In yet another, the restriction endonuclease system comprises a restriction endonuclease unable to cleave duplexes which are ‘first-modified’ or ‘second-unmodified’. In this case, only second fluorophore(s) will be released if the nucleoside triphosphate molecule is modified and only first fluorophore(s) if it is not.

Step (2) results in the first oligonucleotide component of at least one of the duplexes being cleaved into two separate elements one of which bears the first or second fluorophores (as the case may be) and, if employed, the quenchers. This leads to one of three possible statistical outcomes; (i) only exonucleolytically-digestible first oligonucleotide elements bearing first fluorophore(s) are produced, (ii) only exonucleolytically-digestible first oligonucleotide elements bearing second fluorophore(s) are produced or (iii) a mixture of both is obtained.

As mentioned above, at the end of step (2) the component comprised of the second oligonucleotide, one or other of the modified or unmodified nucleotide derived from the nucleoside triphosphate molecule and optionally the third oligonucleotide (for example the fourth oligonucleotide) is in step (3) caused to hybridise to or complex with another corresponding first oligonucleotide molecule in the pair thereby producing a new used-probe duplex. This duplex is then subject to a repeat of steps (2) and (3) thereby releasing further fluorophores in a detectable state and again regenerating the component/fourth oligonucleotide. By this means, steps (2) and (3) iterate to produce a further enhancement in the fluorescence signal; in principle until substantially all of the first oligonucleotide pair has been consumed. As a consequence, the observer sees a much greater enhancement of the fluorescence signal than might otherwise have been obtained. In one embodiment, where no ligation of the nucleotide derived from the nucleoside triphosphate molecule to the third oligonucleotide occurs in step (1) (including but not limited to examples of the method where no third oligonucleotide is employed), step (2) may simply lead to the release of a component comprised of the second oligonucleotide and the nucleotide. In such cases, the new incoming first oligonucleotide may already have a third oligonucleotide attached to it or the third oligonucleotide may be a structural part of it.

In one embodiment, it is preferred that step (2) is carried out at a higher temperature than step (1) and/or that step (3) is carried out at a higher temperature than step (2).

In step (4), the exonucleolytically-digestible first oligonucleotide elements produced in either or both of step (2) and/or each iteration of step (3) are digested by an enzyme exhibiting either 3′-5′ or 5′-3′ exonucleolytic activity depending on which of the two methods is being employed. Thus, as endonucleolysis and subsequent exonucleolysis occurs, the observer sees the onset of and a rapid growth in a fluorescence signal. Step (4) can most effectively be achieved at a temperature in the range 30 to 100° C. It will be appreciated by one of ordinary skill that step (4) can be carried out after iteration step (3) is completed or in parallel as steps (2) and (3) are occurring.

Thereafter, and in step (5), the fluorophores liberated in step (3) or each iteration of steps (2) to (4) are detected and the modification status of the nucleobase attached to the single original nucleoside triphosphate molecule determined by inference; for example in accordance with the outcomes mentioned above. Corresponding detection method are well-known in the art;

for example the reaction medium employed with the method can be interrogated with light from an LED, a laser or like source of high-intensity electromagnetic radiation and any fluorescence generated collected using a photodetector or an fluorescence analyser tuned to the characteristic fluorescence wavelength(s) or wavelength envelope(s) of the various first and second fluorophores. This in turn causes the photodetector to generate a characteristic electrical signal which can be processed and analysed in a computer using known algorithms. In one embodiment the analyte comprises both modified and unmodified nucleoside triphosphate molecules and the method further comprises the step of (6) determining from the results of step (5) the relative proportions of each.

In one particularly preferred embodiment, the method of the present invention is carried out wholly or partially in a stream of microdroplets, at least some of which contain a single nucleoside triphosphate molecule; for example a stream whose ordering reflects the original nucleotide sequence of a progressively digested nucleic acid precursor. Such a method may begin, for example, by inserting the nucleoside triphosphate molecules generated by such progressive digestion one-by-one into a corresponding stream of aqueous microdroplets maintained in an immiscible carrier solvent such as a hydrocarbon or silicone oil to help preserve the ordering. Alternatively, this can be achieved by directly creating the microdroplets downstream of the progressive digestion zone; for example, by causing the reaction medium to emerge from a microdroplet head of suitable dimensions into a flowing stream of the solvent. Alternatively, small aliquots of the reaction medium from the progressive digestion zone can be regularly and sequentially injected into a stream of pre-existing aqueous microdroplets suspended in the solvent. If this latter approach is adopted, each microdroplet may already contain the various components of the probe system(s) together with the enzymes and any other reagents (e.g. buffer) required to effect steps (1) to (4). In yet another approach, the microdroplets created in the former embodiment can be caused to coalesce subsequently with a stream of such pre-existing microdroplets to achieve a similar outcome. In these microdroplet methods, step (5) then preferably involves delivering the microdroplets to a storage area and interrogating each microdroplet to identify the fluorophores liberated; preferably after a period of incubation. Thereafter the results obtained from each microdroplet are assembled into a stream of data characteristic of the original nucleic acid analyte.

To avoid the risk that a given microdroplet contains more than one nucleoside triphosphate molecule it is preferred to release each from the progressive digestion zone at a rate such that each filled microdroplet is separated on average by from 1 to 20 preferably 2 to 10 empty ones. Thereafter the stream of filled and unfilled microdroplets in the solvent is caused to flow along a flow path, suitably a microfluidic flow path, at a rate and in a manner such that they are maintained in a discrete state and do not have the opportunity to coalesce with each other. Suitably the microdroplets employed have a finite diameter less than 100 microns, preferably less than 50 microns, more preferably less than 20 microns and even more preferably less than 15 microns. Most preferably of all their diameters are in the range 2 to 20 microns. In one embodiment, the microdroplet flow rate through the whole system is in the range 50 to 3000 microdroplets per second preferably 100 to 2000.

In a third aspect of the invention there is provided a multi-component biological probe characterised by comprising (a) a pair of single-stranded first oligonucleotides consisting of (i) a pair of single-stranded first oligonucleotides each comprising an exonuclease blocking-site; at least one restriction endonuclease recognition-site located on the 5′ side of the blocking-site; a single nucleotide capture-site located within the recognition-site and respectively first and second fluorophore(s) located on the 5′ side of the recognition-site arranged so as to be substantially undetectable and (ii) at least one second and at least one third single-stranded oligonucleotide each separate from the first oligonucleotide and capable of hybridising to complementary flanking regions on the 3′ and 5′ sides of the capture-site.

In one embodiment, the exonuclease blocking-site is located adjacent the 3′ end of the first oligonucleotide(s). In another embodiment, the exonuclease blocking-site is achieved by making each first oligonucleotide in the pair circular; i.e. a closed-loop.

In a fourth aspect of the invention there is provided a multi-component biological probe characterised by comprising (a) a pair of single-stranded first oligonucleotides consisting of (i) a pair of single-stranded first oligonucleotides each comprising an exonuclease blocking-site; at least one restriction endonuclease recognition-site located on the 3′ side of the blocking-site; a single nucleotide capture-site located within the recognition-site and respectively first and second fluorophore(s) located on the 3′ side of the recognition-site arranged so as to be substantially undetectable and (ii) at least one second and at least one third single-stranded oligonucleotide each separate from the first oligonucleotide and capable of hybridising to complementary flanking regions on the 3′ and 5′ sides of the capture-site.

In one embodiment, the exonuclease blocking-site is located adjacent the 5′ end of the first oligonucleotide(s). In another embodiment, the exonuclease blocking-site is achieved by making each first oligonucleotide in the pair circular; i.e. a closed-loop.

In one embodiment of both of these third and fourth aspects, the various fluorophores on the first oligonucleotides are arranged in close proximity to one another in order to self-quench. In another, the first oligonucleotides include quencher(s) to quench the fluorophores. Suitable fluorophores and quenchers include but are not limited to those described above. In another embodiment the second and third oligonucleotides are connected by a linker-region as explained above; with the linker-region itself preferably being another oligonucleotide region.

As explained above, for the purposes of DNA or RNA sequencing, the biological probes described herein can be assembled into a corresponding biological probe system comprised of a multiplicity of different first oligonucleotide pair types differing only in the nucleobase characteristic of the capture-site and the fluorescence characteristics of the first and second fluorophores used. Optionally, these probes may be employed in association with other biological probes of the type taught in our European patent application 17171168.2. In one embodiment, one, two, three, four or more different first oligonucleotide pair types differing only in the nucleobase characteristic of the capture-site and the first and second fluorophore can be used. For naturally-occurring DNA or RNA, these nucleobases will be comprised of A, G, C, and T or U. In another embodiment, the biological probe system is made manifest as a kit further comprising at least one of a ligase, a polymerase, a restriction endonuclease and an enzyme exhibiting 3′-5′ or 5′-3′ exonucleolytic activity as the case may be.

The invention is now illustrated with reference to the following Example.

### EXAMPLE

### Preparation and Use of a Probe System

Single-stranded first oligonucleotides 1a and 1b are prepared, having the following nucleotide sequences respectively:

5′-TTTCGGGTGAGGTCATGGTCGACAGGTGGGFFQAGATGATGATCAGA
TGTTGCCCTTAGCX-3′
5′-TTTCGAGTGAGGTCATGGTCGACAGGTGGGEEQAGATGATGmATCAG
mATGTTGCCCTTAGCX-3′

wherein A, C, G, and T represent nucleotides bearing the relevant characteristic nucleobase of DNA; F represents a deoxythymidine nucleotide (T) labelled with Atto 700 dye using conventional amine-attachment chemistry; E represents a deoxythymidine nucleotide (T) labelled with Atto 594 dye using conventional amine-attachment chemistry; Q represents a deoxythymidine nucleotide labelled with a BHQ-2 quencher; mA represents an N6-methyl-deoxyadenine nucleotide and X represents an inverted 3′ dT nucleotide. Both first oligonucleotides further comprise a capture region (T nucleotide) at the 43^{rd} base from their 5′ end, selective for capturing deoxyadenine triphosphate nucleotides (dATPs) in a mixture of deoxynucleoside triphosphates (dNTPs), and the recognition sequence for the restriction endonucleases DpnI and DpnII, ‘GATC’.

Another single-stranded oligonucleotide 2, comprising an oligonucleotide region having a sequence complementary to the 3′ region flanking the capture-sites of the two first oligonucleotides, a phosphorothioate linkage at the cutting site of DpnII and a single-stranded oligonucleotide 3, comprising an oligonucleotide region having a sequence complementary to the 5′ region flanking the capture-sites of the two first oligonucleotides with a 5′ phosphate group, and a 3′ inverted dT nucleotide, is also prepared. They have the following nucleotide sequences:

Oligonucleotide 2:
5′-TAAGGGCAACATCT*G-3′
Oligonucleotide 3:
5′-PTCATCATCTAAACCCACCTGTCGAGX-3′

wherein P represents the 5′ phosphate group, X the inverted 3′ dT nucleotide and * the phosphorothioate linkage.

A reaction mixture comprising the probe system is then prepared. It has a composition corresponding to that derived from the following formulation:

20 uL 5× buffer pH 7.9

10 uL oligonucleotide 1a, 250 nM

10 uL oligonucleotide 1b, 250 nM

10 uL oligonucleotide 2, 10 nM

10 uL oligonucleotide 3, 1000 nM

10 U DpnI restriction endonuclease (ex. New England Biolabs Inc.)

10 U DpnII restriction endonuclease (ex. New England Biolabs Inc.)

2.9 U Bst Large Fragment polymerase

2.7 U Platinum Pfx polymerase

6.7 U Thermostable Inorganic Pyrophosphatase

10 uL dATP or N6-methyl-dATP, 1 nM

Water to 100 uL

wherein the 5× buffer comprised the following mixture:

100 uL Trizma Acetate, 1M, pH 8.0

50 uL aqueous Magnesium Acetate, 1M

250 uL aqueous Potassium Acetate, 1M

50 uL Triton X-100 surfactant (10%)

500 μg BSA

Water to 1 ml

Capture of the dATPs or N6-methyl-dATPs is then carried out by incubating the mixture at 37° C. for 10 minutes. The temperature is then held at 37° C. for a further 120 minutes to allow iterated cleaving of the first oligonucleotides. Thereafter, the temperature is then increased to 72° C. for a further 15 minutes to allow digestion of the cleaved first oligonucleotide components bearing the fluorophores and quenchers. The fluorescence intensity of the Atto700 and Atto594 dyes in the reaction mixture is measured using a CLARIOStar microplate reader (ex. BMG Labtech) as the reaction proceeds.

The growth in intensity of fluorescence over the final 15 minute digestion step is monitored in the presence and absence of the dNTP component of the reaction. When no dNTPs are present in the reaction mixture the Atto700 and Atto594 dyes on oligonucleotides 1a and 1b do not exhibit fluorescence to any significant extent. When dATP was present in the detection mixture, DpnII is able to iteratively cleave oligonucleotide 1a, while DpnI is unable to cleave either of the first oligonucleotides, resulting in the generation of signal in the Atto700 channel only after exonucleolysis. When N6-methyl-dATP is present in the detection mixture, DpnI is able to iteratively cleave oligonucleotide 1b, while DpnII is unable to cleave either of the first oligonucleotides, resulting in generation of signal in the Atto594 channel only.

1. A method of detecting whether the nucleobase of a nucleoside triphosphate molecule in an analyte does or does not include a chemical or structural modification comprising the steps of:
(1) reacting the analyte in the presence of a polymerase with a biological probe comprising (a) a pair of single-stranded first oligonucleotides each comprising an exonuclease blocking-site; at least one restriction endonuclease recognition-site located on the 5′ side of the blocking-site; a single nucleotide capture-site located within the endonuclease recognition-site and respectively first and second fluorophore(s) located on the 5′ side of the endonuclease recognition-site arranged so as to be substantially undetectable, and (b) at least one second and optionally at least one third single-stranded oligonucleotide each separate from the first oligonucleotide and capable of hybridising to complementary flanking regions on the 3′ and 5′ sides of the capture-site of one, other or both of the first oligonucleotides in the pair to create a used-probe duplex consisting of (b1) one or other of the first oligonucleotides in the pair and (b2) a component comprised of the second oligonucleotide, one or other of a modified or unmodified nucleotide derived from the nucleoside triphosphate molecule and optionally the third oligonucleotide wherein the duplex is comprised of one of the four possible (b1)/(b2) duplex permutations;
(2) reacting the duplex produced in step (1) with a restriction endonuclease system comprising at least one restriction endonuclease adapted to selectively cleave the (b1) strand at the endonuclease recognition-site to create depending on the presence or absence of the modification in the original nucleoside triphosphate molecule (i) only exonucleolytically-digestible first oligonucleotide elements bearing first fluorophore(s) or (ii) only exonucleolytically-digestible first oligonucleotide elements bearing second fluorophore(s) or (iii) a mixture of exonucleolytically-digestible first oligonucleotide elements respectively bearing first and second fluorophore(s);
(3) creating another used-probe duplex from the (b2) component and another first oligonucleotide in the pair and thereafter iterating steps (2) and (3);
(4) digesting the first oligonucleotide elements with an enzyme having 3′-5′ exonucleolytic activity to produce fluorophore(s) in a detectable state after either or both of steps (2) or (3); and
(5) detecting the fluorophore(s) released in step (4) and inferring from the nature of the fluorescence signal observed whether the original single nucleoside triphosphate molecule was modified or unmodified.

2. A method of detecting whether the nucleobase of a nucleoside triphosphate molecule in an analyte does or does not include a chemical or structural modification comprising the steps of:
(1) reacting the analyte in the presence of a polymerase with a biological probe comprising (a) a pair of single-stranded first oligonucleotides each comprising an exonuclease blocking-site; at least one restriction endonuclease recognition-site located on the 3′ side of the blocking-site; a single nucleotide capture-site located within the endonuclease recognition-site and respectively first and second fluorophore(s) located on the 3′ side of the endonuclease recognition-site arranged so as to be substantially undetectable and (b) at least one second and optionally at least one third single-stranded oligonucleotide each separate from the first oligonucleotide and capable of hybridising to complementary flanking regions on the 3′ and 5′ sides of the capture-site of one, other or both of the first oligonucleotides in the pair to create a used-probe duplex consisting of (b1) one or other of the first oligonucleotides in the pair and (b2) a component comprised of the second oligonucleotide, one or other of a modified or unmodified nucleotide derived from the nucleoside triphosphate molecule and optionally the third oligonucleotide wherein each duplex is comprised of comprising one of the four possible (b1)/(b2) duplex permutations;
(2) reacting the duplex produced in step (1) with a restriction endonuclease system comprised of at least one restriction endonuclease adapted to selectively cleave the (b1) strand at the endonuclease recognition-site to create depending on the presence or absence of the modification in the original nucleoside triphosphate molecule (i) only exonucleolytically-digestible first oligonucleotide elements bearing first fluorophore(s) or (ii) only exonucleolytically-digestible first oligonucleotide elements bearing second fluorophore(s) or (iii) a mixture of exonucleolytically-digestible first oligonucleotide elements respectively bearing first and second fluorophore(s);
(3) creating another used-probe duplex from the (b2) component and another first oligonucleotide in the pair and thereafter iterating steps (2) and (3);
(4) digesting the first oligonucleotide elements with an enzyme having 5′-3′ exonucleolytic activity to produce fluorophore(s) in a detectable state after either or both of steps (2) and (3); and
(5) detecting the fluorophore(s) released in step (4) and inferring from the nature of the fluorescence signal observed whether the original single nucleoside triphosphate molecule was modified or unmodified.

3. The method of claim 1, wherein the restriction endonuclease system comprises (i) a first restriction endonuclease able only to cleave those (b1)-(b2) duplexes comprised of the first oligonucleotide bearing the first fluorophore(s) and the component comprised of the second oligonucleotide, the modified nucleoside triphosphate molecule derived from the analyte and the third oligonucleotide and (ii) a second restriction endonuclease able only to cleave those (b1)-(b2) duplexes comprised of the first oligonucleotide bearing the second fluorophore(s) and the component comprised of the second oligonucleotide, the unmodified nucleoside triphosphate molecule derived from the analyte and the third oligonucleotide.

4. The method of claim 1, wherein the restriction endonuclease system comprises a restriction endonuclease unable to cleave (i) those (b1)-(b2) duplexes comprised of the first oligonucleotide bearing the first fluorophore(s) and the component comprised of the second oligonucleotide, the modified nucleoside triphosphate molecule derived from the analyte and the third oligonucleotide and (ii) those (b1)-(b2) duplexes comprised of the first oligonucleotide bearing the second fluorophore(s) and the component comprised of the second oligonucleotide, the unmodified nucleoside triphosphate molecule derived from the analyte and the third oligonucleotide.

5. The method of claim 1, wherein (i) the restriction endonuclease system comprises a first restriction endonuclease capable of cleaving all of the (b1)-(b2) duplexes and a second restriction endonuclease able to cleave only those (b1)-(b2) duplexes comprised of the first oligonucleotide bearing the second fluorophore(s) and the component comprised of the second oligonucleotide, the unmodified nucleoside triphosphate molecule derived from the analyte and the third oligonucleotide; and (ii) that the recognition-site of the first oligonucleotide bearing the second fluorophore(s) further comprises a first restriction endonuclease blocking-site.

6. The method of claim 1, wherein the restriction endonuclease system comprises a restriction endonuclease able to cleave all (b1)-(b2) duplexes other than those duplexes comprised of the first oligonucleotide bearing the first fluorophore(s) and the component comprised of the second oligonucleotide, the modified nucleoside triphosphate molecule derived from the analyte and the third oligonucleotide.

7. The method of claim 1, wherein the restriction endonuclease system further comprises at least one nicking endonuclease.

8. The method of claim 1, wherein the exonuclease blocking-site is achieved by making each first oligonucleotide a closed-loop.

9. The method of claim 1, wherein step (1) is carried out in the presence of a ligase to create duplexes comprised of one of the first oligonucleotides and a complementary fourth oligonucleotide comprised of the second and third oligonucleotides and the given nucleoside triphosphate molecule derived from the analyte and step (4) further comprises creating another used-probe duplex from the fourth oligonucleotide.

10. The method of claim 1, wherein the second oligonucleotide and the third oligonucleotide are connected by a linker-region which is optionally itself an oligonucleotide.

11. The method of claim 9, wherein the fourth oligonucleotide is resistant to cleavage by the restriction endonuclease.

12. The method of claim 1, wherein either or both of the first oligonucleotides in the pair include one or more quenchers to quench the fluorophore(s).

13. The method of claim 1, wherein the analyte comprises both modified and unmodified single nucleoside triphosphate molecules, and the method further comprises the additional step of determining from the results of step (5) the quantities and relative proportions of each.

14. The method of claim 1, wherein step (2) is carried out at a higher temperature than step (1) and/or that step (3) is carried out at a higher temperature than step (2).

15. The method of claim 1, wherein the nucleoside triphosphate is contained in a corresponding microdroplet and that steps (1) to (4) are carried out in the microdroplet.

16-22. (canceled)

23. The method of claim 2, wherein the restriction endonuclease system comprises (i) a first restriction endonuclease able only to cleave those (b1)-(b2) duplexes comprised of the first oligonucleotide bearing the first fluorophore(s) and the component comprised of the second oligonucleotide, the modified nucleoside triphosphate molecule derived from the analyte and the third oligonucleotide and (ii) a second restriction endonuclease able only to cleave those (b1)-(b2) duplexes comprised of the first oligonucleotide bearing the second fluorophore(s) and the component comprised of the second oligonucleotide, the unmodified nucleoside triphosphate molecule derived from the analyte and the third oligonucleotide.

24. The method of claim 2, wherein the restriction endonuclease system comprises a restriction endonuclease unable to cleave (i) those (b1)-(b2) duplexes comprised of the first oligonucleotide bearing the first fluorophore(s) and the component comprised of the second oligonucleotide, the modified nucleoside triphosphate molecule derived from the analyte and the third oligonucleotide and (ii) those (b1)-(b2) duplexes comprised of the first oligonucleotide bearing the second fluorophore(s) and the component comprised of the second oligonucleotide, the unmodified nucleoside triphosphate molecule derived from the analyte and the third oligonucleotide.

25. The method of claim 2, wherein (i) the restriction endonuclease system comprises a first restriction endonuclease capable of cleaving all of the (b1)-(b2) duplexes and a second restriction endonuclease able to cleave only those (b1)-(b2) duplexes comprised of the first oligonucleotide bearing the second fluorophore(s) and the component comprised of the second oligonucleotide, the unmodified nucleoside triphosphate molecule derived from the analyte and the third oligonucleotide; and (ii) that the recognition-site of the first oligonucleotide bearing the second fluorophore(s) further comprises a first restriction endonuclease blocking-site.

26. The method of claim 2, wherein the restriction endonuclease system comprises a restriction endonuclease able to cleave all (b1)-(b2) duplexes other than those duplexes comprised of the first oligonucleotide bearing the first fluorophore(s) and the component comprised of the second oligonucleotide, the modified nucleoside triphosphate molecule derived from the analyte and the third oligonucleotide.

27. The method of claim 2, wherein the restriction endonuclease system further comprises at least one nicking endonuclease.

28. The method of claim 2, wherein the exonuclease blocking-site is achieved by making each first oligonucleotide a closed-loop.

29. The method of claim 2, wherein step (1) is carried out in the presence of a ligase to create duplexes comprised of one of the first oligonucleotides and a complementary fourth oligonucleotide comprised of the second and third oligonucleotides and the given nucleoside triphosphate molecule derived from the analyte and step (4) further comprises creating another used-probe duplex from the fourth oligonucleotide.

30. The method of claim 2, wherein the second oligonucleotide and the third oligonucleotide are connected by a linker-region which is optionally itself an oligonucleotide.

31. The method of claim 29, wherein the fourth oligonucleotide is resistant to cleavage by the restriction endonuclease.

32. The method of claim 2, wherein either or both of the first oligonucleotides in the pair include one or more quenchers to quench the fluorophore(s).

33. The method of claim 2, wherein the analyte comprises both modified and unmodified single nucleoside triphosphate molecules, and the method further comprises the additional step of determining from the results of step (5) the quantities and relative proportions of each.

34. The method of claim 2, wherein step (2) is carried out at a higher temperature than step (1) and/or that step (3) is carried out at a higher temperature than step (2).

35. The method of claim 2, wherein the nucleoside triphosphate is contained in a corresponding microdroplet and that steps (1) to (4) are carried out in the microdroplet.


SEQUENCE LISTING (4 sequences)

SEQ ID NO: 1 - DNA, 61 bp, Artificial sequence
  single-stranded first oligonucleotide 1a
  T labelled with Atto 700 dye (31)..(31): F represents a deoxythymidine nucleotide (T) labelled with Atto 700 dye
  T labelled with Atto 700 dye (32)..(32): F represents a deoxythymidine nucleotide (T) labelled with Atto 700 dye
  T labelled with a BHQ-2 quencher (33)..(33): Q represents a deoxythymidine nucleotide labelled with a BHQ-2 quencher
  inverted 3′ dT nucleotide (61)..(61): X represents an inverted 3′ dT nucleotide
tttcgggtgaggtcatggtcgacaggtgggtttagatgatgatcagatgttgcccttagct

SEQ ID NO: 2 - DNA, 61 bp, Artificial sequence
  single-stranded oligonucleotide 1b
  (T) labelled with Atto 700 dye (31)..(31): F represents a deoxythymidine nucleotide (T) labelled with Atto 700 dye
  (T) labelled with Atto 700 dye (32)..(32): F represents a deoxythymidine nucleotide (T) labelled with Atto 700 dye
  (T) labelled with a BHQ-2 quencher (33)..(33): Q represents a deoxythymidine nucleotide labelled with a BHQ-2 quencher
  inverted 3′ dT nucleotide (61)..(61): X represents an inverted 3′ dT nucleotide
tttcgagtgaggtcatggtcgacaggtgggtttagatgatgatcagatgttgcccttagct

SEQ ID NO: 3 - DNA, 15 bp, Artificial sequence
  single-stranded oligonucleotide 2
  phosphorothioate linkage (14)..(15): phosphorothioate linkage
taagggcaacatctg

SEQ ID NO: 4 - DNA, 26 bp, Artificial sequence
  single-stranded oligonucleotide 3
  5′ phosphate group (1)..(1): 5′ phosphate group
  X the inverted 3′ dT nucleotide (26)..(26): X the inverted 3′ dT nucleotide
tcatcatctaaacccacctgtcgagt